SEQUENCE LISTING (4 sequences)

SEQ ID NO: 1 - protein, 494 aa, Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKIFLPAKTEEDIFAHLGLDYIDPWQRNA

SEQ ID NO: 2 - protein, 509 aa, Bos taurus
MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 3 - protein, 530 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

SEQ ID NO: 4 - protein, 494 aa, Homo sapiens
MLPKRRRARVGSPSGDAASSTPPSTRFPGVAIYLVEPRMGRSRRAFLTGLARSKGFRVLDACSSEATHVVMEETSAEEAVSWQERRMAAAPPGCTPPALLDISWLTESLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

(19) United States
(12) Patent Application Publication
(10) Pub. No.: US 2023/0357730 A1
(43) Pub. Date: Nov. 9, 2023

(54) Modified Terminal Deoxynucleotidyl Transferase (TdT) Enzymes

(71) Applicant: Nuclera Ltd, Cambridge (GB)

(72) Inventors: Gordon Ross McInroy, Cambridge (GB); Ian Haston Cook, Cambridge (GB); Michael Chun Hao Chen, Cambridge (GB); Sihong Chen, Cambridge (GB)

(21) Appl. No.: 18/017,704

(22) PCT Filed: Aug. 4, 2021
(86) PCT No.: PCT/GB2021/052011

§ 371 Date: Jan. 24, 2023

(30) Foreign Application Priority Data
Aug. 4, 2020 (GB) 2012093.7
Aug. 12, 2020 (GB) 2012542.3

(51) Int. Cl. C12N 9/12; C12P 19/34

(52) U.S. Cl. CPC C12N 9/1241; C12P 19/34; C12Y 207/07031

(57) ABSTRACT

The invention relates to the use of specific terminal deoxynucleotidyl transferase (TdT) enzymes or the homologous amino acid sequence of PoIµ, PoIβ, PoIλ, and PoIθ of any species or the homologous amino acid sequence of X family polymerases of any species in a method of nucleic acid synthesis, to methods of synthesizing nucleic acids, and to the use of kits comprising said enzymes in a method of nucleic acid synthesis. The invention also relates to the use of terminal deoxynucleotidyl transferases or homologous enzymes and 3′-blocked nucleoside triphosphates in a method of template independent nucleic acid synthesis.

### FIELD OF THE INVENTION

The invention relates to the use of specific terminal deoxynucleotidyl transferase (TdT) enzymes or the homologous amino acid sequence of Polµ, Polβ, Polλ, and Polθ of any species or the homologous amino acid sequence of X family polymerases of any species in a method of nucleic acid synthesis, to methods of synthesizing nucleic acids, and to the use of kits comprising said enzymes in a method of nucleic acid synthesis. The invention also relates to the use of terminal deoxynucleotidyl transferases or homologous enzymes and 3′-blocked nucleoside triphosphates in a method of template independent nucleic acid synthesis.

### BACKGROUND OF THE INVENTION

Nucleic acid synthesis is vital to modern biotechnology. The rapid pace of development in the biotechnology arena has been made possible by the scientific community’s ability to artificially synthesise DNA, RNA and proteins.

Artificial DNA synthesis allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. Despite being a mature technology, it is highly challenging to synthesise a DNA strand greater than 200 nucleotides in length in viable yield, and most DNA synthesis companies only offer up to 120 nucleotides routinely. In comparison, an average protein-coding gene is of the order of 2000-3000 contiguous nucleotides, a chromosome is at least a million contiguous nucleotides in length and an average eukaryotic genome numbers in the billions of nucleotides. In order to prepare nucleic acid strands thousands of base pairs in length, all major gene synthesis companies today rely on variations of a ‘synthesise and stitch’ technique, where overlapping 40-60-mer fragments are synthesised and stitched together by enzymatic copying and extension. Current methods generally allow up to 3 kb in length for routine production.

The reason DNA cannot be synthesised beyond 120-200 nucleotides at a time is due to the current methodology for generating DNA, which uses synthetic chemistry (i.e., phosphoramidite technology) to couple a nucleotide one at a time to make DNA. Even if the efficiency of each nucleotide-coupling step is 99% efficient, it is mathematically impossible to synthesise DNA longer than 200 nucleotides in acceptable yields. The Venter Institute illustrated this laborious process by spending 4 years and 20 million USD to synthesise the relatively small genome of a bacterium.

Known methods of DNA sequencing use template-dependent DNA polymerases to add 3′-reversibly terminated nucleotides to a growing double-stranded substrate. In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

Various attempts have been made to use a terminal deoxynucleotidyl transferase for de novo single-stranded DNA synthesis. Uncontrolled de novo single-stranded DNA synthesis, as opposed to controlled, takes advantage of TdT’s deoxynucleoside 5′-triphosphate (dNTP) 3′- tailing properties on single-stranded DNA to create, for example, homopolymeric adaptor sequences for next-generation sequencing library preparation. In controlled extensions, reversible deoxynucleoside 5′-triphosphate termination technology needs to be employed to prevent uncontrolled addition of dNTPs to the 3′-end of a growing DNA strand. The development of a controlled single-stranded DNA synthesis process through TdT would be invaluable to in situ DNA synthesis for gene assembly or hybridization microarrays as it removes the need for an anhydrous environment and allows the use of various polymers incompatible with organic solvents.

However, TdT has not been shown to efficiently add nucleoside triphosphates containing 3′-O- reversibly terminating moieties for building up a nascent single-stranded DNA chain necessary for a de novo synthesis cycle. A 3′-O- reversible terminating moiety would prevent a terminal transferase like TdT from catalysing the nucleotide transferase reaction between the 3′-end of a growing DNA strand and the 5′-triphosphate of an incoming nucleoside triphosphate.

There is therefore a need to identify modified terminal deoxynucleotidyl transferases that readily incorporate 3′-O- reversibly terminated nucleotides. Said modified terminal deoxynucleotidyl transferases can be used to incorporate 3′-O- reversibly terminated nucleotides in a fashion useful for biotechnology and single-stranded DNA synthesis processes in order to provide an improved method of nucleic acid synthesis that is able to overcome the problems associated with currently available methods. The applicants have previously identified novel enzymes in application PCT/GB2020/050247. Described herein are further improved enzymes.

### BRIEF DESCRIPTION OF THE FIGURES

FIG. 1. Mutations that increase the TdT activity of non-templated de novo nucleic acid synthesis through the use of 3′-reversibly terminated nucleoside 5′-triphosphates. N+17 multi-cycling enzymatic DNA synthesis experiment. Synthesised DNA libraries were subsequently prepared and analysed by Illumina next-generation sequencing (iSeq). Percent perfect (i.e., percentage of total quantity of reads matching the intended synthesised sequence) indicated 14 variants mutated at P422 and/or R442 as better than the parental control. Note the amino acid numbering is for the truncated region (P282 is P422 and R302 is R442).

FIG. 2. Sequence alignment of selected orthologs of wild-type terminal deoxynucleotidyl transferases using the Clustal Omega multiple sequence alignment program provided by the European Molecular Biology Laboratory (EMBL) multiple sequence alignment site.

FIG. 3. Mutations that increase the TdT activity of non-templated de novo nucleic acid synthesis through the use of 3′-reversibly terminated nucleoside 5′-triphosphates. N+17 multi-cycling enzymatic DNA synthesis experiment. Synthesised DNA libraries were subsequently prepared and analysed by Illumina next-generation sequencing (iSeq). Percent perfect (i.e., percentage of total quantity of reads matching the intended synthesised sequence) indicated that L265P K392M is the best performer of this validation screen. Note the amino acid numbering is for the truncated sequences (L126 is L265 etc).

FIGS. 4. Mutations that increase the TdT activity of non-templated de novo nucleic acid synthesis through the use of 3′-reversibly terminated nucleoside 5′-triphosphates. N+17 multi-cycling enzymatic DNA synthesis experiment. Synthesised DNA libraries were subsequently prepared and analysed by Illumina next-generation sequencing (iSeq). Percent perfect (i.e., percentage of total quantity of reads matching the intended synthesised sequence) indicated that P282S, R302Q and E245N is the best performer of this validation screen. Note the amino acid numbering is for the truncated sequences (E245 is E385 etc). FIG. 4a shows the number of perfect full length reads. FIG. 4b shows the efficiency per coupling cycle. Date shown is the figures is below:

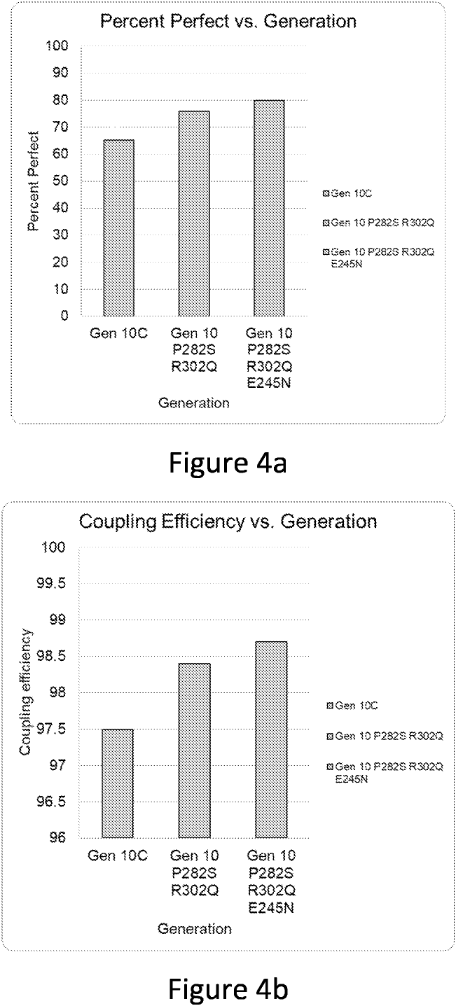

| Generation | Percent Perfect | Coupling Efficiency |
|---|---|---|
| Gen 10C | 65.1 | 97.5 |
| Gen 10 P282S R302Q | 75.8 | 98.4 |
| Gen 10 P282S R302Q E245N | 80.0 | 98.7 |

### SUMMARY OF THE INVENTION

Described herein are modified terminal deoxynucleotidyl transferase (TdT) enzymes or the homologous amino acid sequence of Polµ, Polβ, Polλ, and Polθ of any species or the homologous amino acid sequence of X family polymerases of any species. Terminal transferase enzymes are ubiquitous in nature and are present in many species. Many known TdT sequences have been reported in the NCBI database http://www.ncbi.nlm.nih.gov/.

| | |
|---|---|
| GI Number | Species http://www.ncbi.nlm.nih.gov/ |
| gi\|768 | Bos taurus |

| | |
|---|---|
| gi\|460163 | Gallus gallus |
| gi\|494987 | Xenopus laevis |
| gi\|1354475 | Oncorhynchus mykiss |
| gi\|2149634 | Monodelphis domestica |
| gi\|12802441 | Mus musculus |
| gi\|28852989 | Ambystoma mexicanum |
| gi\|38603668 | Takifugu rubripes |
| gil40037389 | Raja eglanteria |
| gil40218593 | Ginglymostoma cirratum |
| gi\|46369889 | Danio rerio |
| gi\|73998101 | Canis lupus familiaris |
| gi\|139001476 | Lemur catta |
| gi\|139001490 | Microcebus murinus |
| gi\|139001511 | Otolemur garnettii |
| gi\|148708614 | Mus musculus |
| gi\|149040157 | Rattus norvegicus |
| gi\|149704611 | Equus caballus |
| gi\|164451472 | Bos taurus |
| gi\|169642654 | Xenopus (Silurana) tropicalis |
| gi\|291394899 | Oryctolagus cuniculus |
| gi\|291404551 | Oryctolagus cuniculus |
| gi\|301763246 | Ailuropoda melanoleuca |
| gi\|311271684 | Sus scrofa |
| gi\|327280070 | Anolis carolinensis |
| gi\|334313404 | Monodelphis domestica |
| gi\|344274915 | Loxodonta africana |
| gi\|345330196 | Ornithorhynchus anatinus |
| gi\|348588114 | Cavia porcellus |
| gi\|351697151 | Heterocephalus glaber |
| gi\|355562663 | Macaca mulatta |
| gi\|395501816 | Sarcophilus harrisii |
| gi\|395508711 | Sarcophilus harrisii |
| gi\|395850042 | Otolemur garnettii |
| gi\|397467153 | Pan paniscus |

| | |
|---|---|
| gi\|403278452 | Saimiri boliviensis boliviensis |
| gi\|410903980 | Takifugu rubripes |
| gi\|410975770 | Felis catus |
| gi\|432092624 | Myotis davidii |
| gi\|432113117 | Myotis davidii |
| gi\|444708211 | Tupaia chinensis |
| gi\|460417122 | Pleurodeles waltI |
| gi\|466001476 | Orcinus orca |
| gi\|471358897 | Trichechus manatus latirostris |
| gi\|478507321 | Ceratotherium simum simum |
| gi\|478528402 | Ceratotherium simum simum |
| gi\|488530524 | Dasypus novemcinctus |
| gi\|499037612 | Maylandia zebra |
| gi\|504135178 | Ochotona princeps |
| gi\|505844004 | Sorex araneus |
| gi\|505845913 | Sorex araneus |
| gi\|507537868 | Jaculus jaculus |
| gi\|507572662 | Jaculus jaculus |
| gi\|507622751 | Octodon degus |
| gi\|507640406 | Echinops telfairi |
| gi\|507669049 | Echinops telfairi |
| gi\|507930719 | Condylura cristata |
| gi\|507940587 | Condylura cristata |
| gi\|511850623 | Mustela putorius furo |
| gi\|512856623 | Xenopus (Silurana) tropicalis |
| gi\|512952456 | Heterocephalus glaber |
| gi\|524918754 | Mesocricetus auratus |
| gi\|527251632 | Melopsittacus undulatus |
| gi\|528493137 | Danio rerio |
| gi\|528493139 | Danio rerio |
| gi\|529438486 | Falco peregrinus |
| gi\|530565557 | Chrysemys picta bellii |
| gi\|532017142 | Microtus ochrogaster |
| gi\|532099471 | Ictidomys tridecemlineatus |

| | |
|---|---|
| gi\|533166077 | Chinchilla lanigera |
| gi\|533189443 | Chinchilla lanigera |
| gi\|537205041 | Cricetulus griseus |
| gi\|537263119 | Cricetulus griseus |
| gi\|543247043 | Geospiza fortis |
| gi\|543351492 | Pseudopodoces humilis |
| gi\|543731985 | Columba livia |
| gi\|544420267 | Macaca fascicularis |
| gi\|545193630 | Equus caballus |
| gi\|548384565 | Pundamilia nyererei |
| gi\|551487466 | Xiphophorus maculatus |
| gi\|551523268 | Xiphophorus maculatus |
| gi\|554582962 | Myotis brandtii |
| gi\|554588252 | Myotis brandtii |
| gi\|556778822 | Pantholops hodgsonii |
| gi\|556990133 | Latimeria chalumnae |
| gi\|557297894 | Alligator sinensis |
| gi\|558116760 | Pelodiscus sinensis |
| gi\|558207237 | Myotis lucifugus |
| gi\|560895997 | Camelus ferus |
| gi\|560897502 | Camelus ferus |
| gi\|562857949 | Tupaia chinensis |
| gi\|562876575 | Tupaia chinensis |
| gi\|564229057 | Alligator mississippiensis |
| gi\|564236372 | Alligator mississippiensis |
| gi\|564384286 | Rattus norvegicus |
| gi\|573884994 | Lepisosteus oculatus |

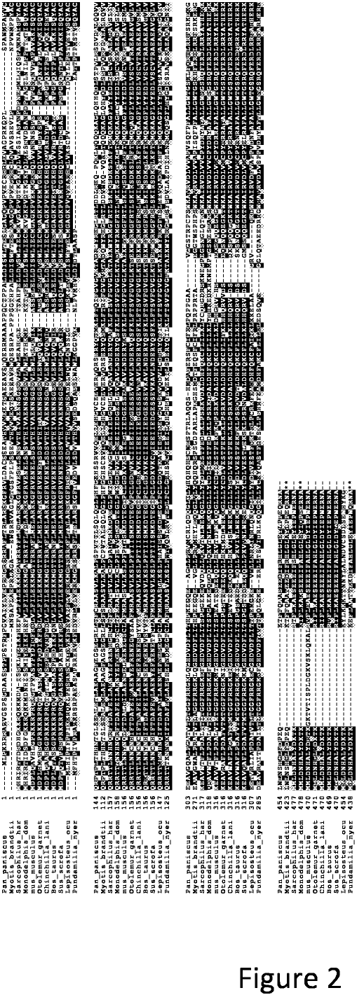

The sequences of the various described terminal transferases show some regions of highly conserved sequence, and some regions which are highly diverse between different species. A sequence alignment for sequences from a selection of species is shown in FIG. 2.

The inventors have modified the terminal transferase from Lepisosteus oculatus TdT (spotted gar) (shown as SED ID 1). However the corresponding modifications can be introduced into the analagous terminal transferase sequences from any other species, including the sequences listed above in the various NCBI entries, including those shown in FIG. 2 or truncated versions thereof.

The amino acid sequence of the spotted gar (Lepisosteus oculatus) is shown below (SEQ ID 1)

MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

An engineered variant of this sequence was previously identified as SEQ ID NO 8 in publication WO2016/128731. Further engineered Improvements to this published sequence are described in PCT/GB2020/050247. The modified sequences disclosed herein are further improved alterations over the sequences disclosed in the prior art. WO2016/128731 SEQ ID NO 2 is a “mis-annotated” wild-type gar sequence.

All amino acid numbering is in reference to sequence ID 1, the full length sequence of 494 amino acids. Applicants use truncations of the full length sequence which retain activity, and thus the truncations, being fewer amino acids, will have different numbering.

SEQ ID NO 8 in publication WO2016/128731 is shown below with the engineered mutations identified:

MLHIPIFPPIKKRQKLPESRNSCKYEVKFSEVAIFLVERKMGSSRRKFLT
NLARSKGFRIEDVLSDAVTHVVAENNSADELLQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LRTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

The inventors have identified various amino acids modifications in the amino acid sequence having improved properties. The modifications described herein improve the ability to incorporate nucleotides with modifications; these modifications include modifications at the 3′-position of the sugar and modifications to the base.

Described herein are modified terminal deoxynucleotidyl transferase (TdT) enzymes comprising amino acid modifications when compared to a wild type sequence SEQ ID NO 1 or a truncated version thereof or the homologous amino acid sequence of a terminal deoxynucleotidyl transferase (TdT) enzyme in other species or the homologous amino acid sequence of Polµ, Polβ, Polλ, and Polθ of any species or the homologous amino acid sequence of X family polymerases of any species, wherein the amino acid is modified at one or more of the amino acids:

T160, E174, C179, M183, A195, S198, D210, Q211, Q224, S245, R259, H263, L265,
A273, H275, L285, A293, G303, Q304, L312, A314, C331, V335, M344, V348, R357,
D368, I369, E385, M387, D388, F390, K392, F394, K401, A404, P422, V424, E441,
R442, R445, K453, N458, K464, or D488.

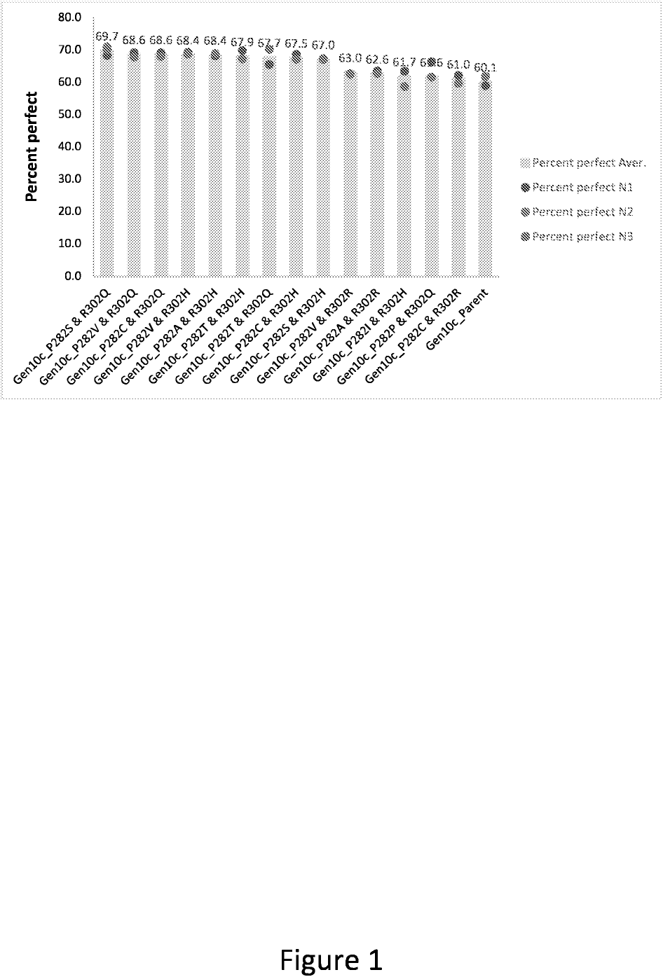

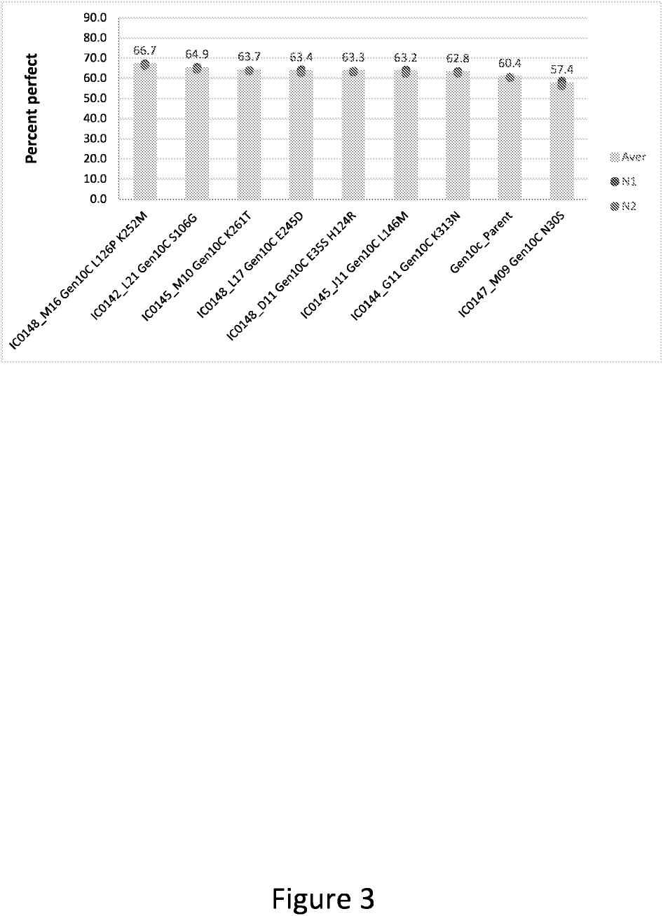

Modifications which improve the incorporation of modified nucleotides can be at one or more of the selected positions shown below. Positions were selected according to mutation data (FIGS. 1 and 3) and sequence alignment (FIG. 2).

MLHIPIFPPIKKRQKLPESRNSCKYEVKFSEVAIFLVERKMGSSRRKFLT
NLARSKGFRIEDVLSDAVTHVVAEDNSADELWQWLQNSSLGDLSKIEVLD
ISWFTECMGAGKPVQVEARHCLVKSCPVIDQYLEPSTVETVSQYACQRRT
TMENHNQIFTDAFAILAENAEFNESEGPCLAFMRAASLLKSLPHAISSSK
DLEGLPCLGDQTKAVIEDILEYGQCSKVQDVLCDDRYQTIKLFTSVFGVG
LKTAEKWYRKGFHSLEEVQADNAIHFTKMQKAGFLYYDDISAAVCKAEAQ
AIGQIVEETVRLIAPDAIVTLTGGFRRGKECGHDVDFLITTPEMGKEVWL
LNRLINRLQNQGILLYYDIVESTFDKTRLPCRKFEAMDHFQKCFAIIKLK
KELAAGRVQKDWKAIRVDFVAPPVDNFAFALLGWTGSRQFERDLRRFARH
ERKMLLDNHALYDKTKKIFLPAKTEEDIFAHLGLDYIDPWQRNA

Described herein is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid positions T160, E174, C179, M183, A195, S245, H263, L265, L285, A293, D368, E385, M387, D388, K392, F394, K401, P422, E441, R442, K453, N458 or D488 of the sequence of SEQ ID NO 1 or the homologous regions in other species.

Described herein is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid positions T160, E174, C179, M183, A195, S198, D210, Q211, Q224, S245, R259, H263, L265, A273, H275, L285, A293, G303, Q304, L312, A314, C331, V335, M344, V348, R357, D368, I369, E385, M387, D388, F390, K392, F394, K401, A404, P422, V424, E441, R442, R445, K453, N458, K464, or D488 of the sequence of SEQ ID NO 1 or the homologous regions in other species.

References to particular sequences include truncations thereof. Included herein are modified terminal deoxynucleotidyl transferase (TdT) enzyme comprising at least one amino acid modification when compared to a wild type sequence SEQ ID NO 1 or a truncated version thereof, or the homologous amino acid sequence of a terminal deoxynucleotidyl transferase (TdT) enzyme in other species, wherein the modification is selected from one or more of the amino acid of the sequence of SEQ ID NO 1 or the homologous regions in other species.

Truncated proteins may include at least the region shown below including one or more of the relevant modifications.

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

Described herein is a modified terminal deoxynucleotidyl transferase (TdT) enzyme comprising at least the sequence:

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

or the homologous regions in other species, wherein the sequence has one or more amino acid modifications in one or more of the amino acid positions T160, E174, C179, M183, A195, S245, H263, L265, L285, A293, D368, E385, M387, D388, K392, F394, K401, P422, E441, R442, K453, N458 or D488 of the full length sequence.

For reference, the modifications are shown in the truncated sequence:

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

Homologous refers to protein sequences between two or more proteins that possess a common evolutionary origin, including proteins from superfamilies in the same species of organism as well as homologous proteins from different species. Such proteins (and their encoding nucleic acids) have sequence homology, as reflected by their sequence similarity, whether in terms of percent identity or by the presence of specific residues or motifs and conserved positions. A variety of protein (and their encoding nucleic acid) sequence alignment tools may be used to determine sequence homology. For example, the Clustal Omega multiple sequence alignment program provided by the European Molecular Biology Laboratory (EMBL) can be used to determine sequence homology or homologous regions.

Improved sequences as described herein can contain two or more of the aforementioned modifications, namely, for example,
a. a first modification at position C179 of the sequence of SEQ ID NO 1 or the homologous region in other species; and
b. a second modification at position D488 of the sequence of SEQ ID NO 1 or the homologous regions in other species or a truncated sequence.

Improved sequences as described herein can contain three or more of the aforementioned modifications, namely, for example,
a. a first modification at position E385 of the sequence of SEQ ID NO 1 or the homologous region in other species; and
b. a second modification at position P422 of the sequence of SEQ ID NO 1 or the homologous regions in other species or a truncated sequence; and
c. a third modification at position R442 of the sequence of SEQ ID NO 1 or the homologous regions in other species or a truncated sequence; and

Improved sequences as described herein can contain one of the aforementioned modifications, namely,
a modification at T160,
a modification at E174,
a modification at C179,
a modification at M183,
a modification at A195
a modification at S245,
a modification at H263,
a modification at L265,
a modification at L285,
a modification at A293,
a modification at D368,
a modification at E385,
a modification at M387,
a modification at D388,
a modification at K392,
a modification at F394,
a modification at K401,
a modification at P422,
a modification at E441,
a modification at R442,
a modification at K453,
a modification at N458,
a modification at D488.

Improved sequences as described herein can contain one of the aforementioned modifications, namely
a modification at T160,
a modification at E174,
a modification at C179,
a modification at M183,
a modification at A195,
a modification at S198,
a modification at D210,
a modification at Q211,
a modification at Q224,
a modification at S245,
a modification at R259,
a modification at H263,
a modification at L265,
a modification at A273
a modification at H275
a modification at L285,
a modification at A293,
a modification at G303,
a modification at Q304,
a modification at L312,
a modification at A314,
a modification at C331,
a modification at V335,
a modification at M344,
a modification at V348,
a modification at R357,
a modification at D368,
a modification at I369,
a modification at E385,
a modification at M387,
a modification at D388,
a modification at F390,
a modification at K392,
a modification at F394,
a modification at K401,
a modification at A404,
a modification at P422,
a modification at V424,
a modification at E441,
a modification at R442,
a modification at R445,
a modification at K453,
a modification at N458,
a modification at K464,
a modification at D488.

As a comparison with other species, the sequence of Bos taurus (cow) TdT is shown below:

MDPLCTASSGPRKKRPRQVGASMASPPHDIKFQNLVLFILEKKMGTTRRN
FLMELARRKGFRVENELSDSVTHIVAENNSGSEVLEWLQVQNIRASSQLE
LLDVSWLIESMGAGKPVEITGKHQLVVRTDYSATPNPGFQKTPPLAVKKI
SQYACQRKTTLNNYNHIFTDAFEILAENSEFKENEVSYVTFMRAASVLKS
LPFTIISMKDTEGIPCLGDKVKCIIEEIIEDGESSEVKAVLNDERYQSFK
LFTSVFGVGLKTSEKWFRMGFRSLSKIMSDKTLKFTKMQKAGFLYYEDLV
SCVTRAEAEAVGVLVKEAVWAFLPDAFVTMTGGFRRGKKIGHDVDFLITS
PGSAEDEEQLLPKVINLWEKKGLLLYYDLVESTFEKFKLPSRQVDTLDHF
QKCFLILKLHHQRVDSSKSNQQEGKTWKAIRVDLVMCPYENRAFALLGWT
GSRQFERDIRRYATHERKMMLDNHALYDKTKRVFLKAESEEEIFAHLGLD
YIEPWERNA

Corresponding amino acids
T160 = T169
E174 = E183
C179 = Y188
M183 = M192
A195 = T204
S245 = S254
H263 = R272
L265 = L274
L285 = L294
A295 = C302
D368 = D378
E385 = D395
M387 = L397
D388 = D398
K392 = K402
F394 = F404
K401 = H411
P422 = C437
E441 = E456
R442 = R460
K453 = K468
N458 = N473
D488 = E503

Corresponding amino acids
T160 = T169
E174 = E183
C179 = Y188
M183 = M192
A195 = T204
S198 = S207
D210 = D219
Q211 = K220
Q224, = E233
S245 = S254
R259 = R268
H263 = R272
L265 = L274
A273 = T282
H275 = K284
L285 = L294
A293 = C302
A295 = T304
G303 = G312
Q304 = V313
L312 = A321
A314 = L323
C331 = 1340
V335 = V344
M344 = S353
V348 = E358
R357 = L367
D368 = D378
I369 = L379
E385 = D395
M387 = L397
D388 = D398
F390 = F400
K392 = K402
F394 = F404
K401 = H411
A404 = V414
P422 = C437
V424 = Y439
E441 = E456
R442 = R457
R445 = R460
K453 = K468
N458 = N473
K464 = K479
D488 = E503

The amino acid positions are highlighted below

MDPLCTASSGPRKKRPRQVGASMASPPHDIKFQNLVLFILEKKMGTTRRN
FLMELARRKGFRVENELSDSVTHIVAENNSGSEVLEWLQVQNIRASSQLE
LLDVSWLIESMGAGKPVEITGKHQLVVRTDYSATPNPGFQKTPPLAVKKI
SQYACQRKTTLNNYNHIFTDAFEILAENSEFKENEVSYVTFMRAASVLKS
LPFTIISMKDTEGIPCLGDKVKCIIEEIIEDGESSEVKAVLNDERYQSFK
LFTSVFGVGLKTSEKWFRMGFRSLSKIMSDKTLKFTKMQKAGFLYYEDLV
SCVTRAEAEAVGVLVKEAVWAFLPDAFVTMTGGFRRGKKIGHDVDFLITS
PGSAEDEEQLLPKVINLWEKKGLLLYYDLVESTFEKFKLPSRQVDTLDHH
FQKCFLILKLHHQRVDSSKSNQQEGKTWKAIRVDLVMCPYENRAFALLGW
TGSRQFERDIRRYATHERKMMLDNHALYDKTKRVFLKAESEEEIFAHLGL
DYIEPWERNA

As a comparison with other species, the sequence of Mus musculus (mouse) TdT is shown below:

MDPLQAVHLGPRKKRPRQLGTPVASTPYDIRFRDLVLFILEKKMGTTRRA
FLMELARRKGFRVENELSDSVTHIVAENNSGSDVLEWLQLQNIKASSELE
LLDISWLIECMGAGKPVEMMGRHQLVVNRNSSPSPVPGSQNVPAPAVKKI
SQYACQRRTTLNNYNQLFTDALDILAENDELRENEGSCLAFMRASSVLKS
LPFPITSMKDTEGIPCLGDKVKSIIEGIIEDGESSEAKAVLNDERYKSFK
LFTSVFGVGLKTAEKWFRMGFRTLSKIQSDKSLRFTQMQKAGFLYYEDLV
SCVNRPEAEAVSMLVKEAVVTFLPDALVTMTGGFRRGKMTGHDVDFLITS
PEATEDEEQQLLHKVTDFWKQQGLLLYCDILESTFEKFKQPSRKVDALDH
FQKCFLILKLDHGRVHSEKSGQQEGKGWKAIRVDLVMCPYDRRAFALLGW
TGSRQFERDLRRYATHERKMMLDNHALYDRTKGKTVTISPLDGKVSKLQK
ALRVFLEAESEEEIFAHLGLDYIEPWERNA

Modifications which improve the incorporation of modified nucleotides can be at one or more of selected positions shown below. The second modification can be selected from one or more of the amino acid positions C179, E488, E441, M183 and N458 shown highlighted in the sequence below.

MDPLQAVHLGPRKKRPRQLGTPVASTPYDIRFRDLVLFILEKKMGTTRRA
FLMELARRKGFRVENELSDSVTHIVAENNSGSDVLEWLQLQNIKASSELE
LLDISWLIECMGAGKPVEMMGRHQLVVNRNSSPSPVPGSQNVPAPAVKKI
SQYACQRRTTLNNYNQLFTDALDILAENDELRENEGSCLAFMRASSVLKS
LPFPITSMKDTEGIPCLGDKVKSIIEGIIEDGESSEAKAVLNDERYKSFK
LFTSVFGVGLKTAEKWFRMGFRTLSKIQSDKSLRFTQMQKAGFLYYEDLV
SCVNRPEAEAVSMLVKEAVVTFLPDALVTMTGGFRRGKMTGHDVDFLITS
PEATEDEEQQLLHKVTDFWKQQGLLLYCDILESTFEKFKQPSRKVDALDH
FQKCFLILKLDHGRVHSEKSGQQEGKGWKAIRVDLVMCPYDRRAFALLGW
TGSRQFERDLRRYATHERKMMLDNHALYDRTKGKTVTISPLDGKVSKLQK
ALRVFLEAESEEEIFAHLGLDYIEPWERNA

Thus by a process of aligning sequences, it is immediately apparent which regions in the sequences of terminal transferases from other species correspond to the sequences described herein with respect to the spotted gar sequence shown in SEQ ID NO 1.

Sequence homology extends to all modified or wild-type members of family X polymerases, such as DNA Polµ (also known as DNA polymerase mu or POLM), DNA Polβ (also known as DNA polymerase beta or POLB), and DNA Polλ (also known known as DNA polymerase lambda or POLL). It is well known in the art that all family X member polymerases, of which TdT is a member, either have terminal transferase activity or can be engineered to gain terminal transferase activity akin to terminal deoxynucleotidyl transferase (Biochim Biophys Acta. 2010 May; 1804(5): 1136-1150). For example, when the following human TdT loop1 amino acid sequence

...ESTFEKLRLPSRKVDALDHF...

was engineered to replace the following human Polµ amino acid residues

...HSCCESPTRLAQQSHMDAF...,

the chimeric human Polµ containing human TdT loop1 gained robust terminal transferase activity (Nucleic Acids Res. 2006 Sep; 34(16): 4572-4582).

Furthermore, it was generally demonstrated in U.S. Pat. Application No. 2019/0078065 that family X polymerases when engineered to contain TdT loop1 chimeras could gain robust terminal transferase activity. Additionally, it was demonstrated that TdT could be converted into a template-dependent polymerase through specific mutations in the loop1 motif (Nucleic Acids Research, June 2009, 37(14):4642-4656). As it has been shown in the art, family X polymerases can be trivially modified to either display template-dependent or template-independent nucleotidyl transferase activities. Therefore, all motifs, regions, and mutations demonstrated in this patent can be trivially extended to modified X family polymerases to enable modified X family polymerases to incorporate 3′-modified nucleotides, reversibly terminated nucleotides, and modified nucleotides in general to effect methods of nucleic acid synthesis.

As a comparison with other family X polymerases, the human Polµ sequence is shown below:

MLPKRRRARVGSPSGDAASSTPPSTRFPGVAIYLVEPRMGRSRRAFLTGL
ARSKGFRVLDACSSEATHVVMEETSAEEAVSWQERRMAAAPPGCTPPALL
DISWLTESLGAGQPVPVECRHRLEVAGPRKGPLSPAWMPAYACQRPTPLT
HHNTGLSEALEILAEAAGFEGSEGRLLTFCRAASVLKALPSPVTTLSQLQ
GLPHFGEHSSRVVQELLEHGVCEEVERVRRSERYQTMKLFTQIFGVGVKT
ADRWYREGLRTLDDLREQPQKLTQQQKAGLQHHQDLSTPVLRSDVDALQQ
VVEEAVGQALPGATVTLTGGFRRGKLQGHDVDFLITHPKEGQEAGLLPRV
MCRLQDQGLILYHQHQHSCCESPTRLAQQSHMDAFERSFCIFRLPQPPGA
AVGGSTRPCPSWKAVRVDLVVAPVSQFPFALLGWTGSKLFQRELRRFSRK
EKGLWLNSHGLFDPEQKTFFQAASEEDIFRHLGLEYLPPEQRNA

Thus by a process of aligning sequences, it is immediately apparent which positions in the sequences of all family X polymerases from any species correspond to the sequences described herein with respect to the spotted gar sequence shown in SEQ ID NO 1.

Furthermore, the A family polymerase, DNA Polθ (also known as DNA polymerase theta or POLQ) was demonstrated to display robust terminal transferase capability (eLife. 2016; 5: e13740). DNA Polθ was also demonstrated to be useful in methods of nucleic acid synthesis (GB patent application no. 2553274). In U.S. Pat. Application No. 2019/0078065, it was demonstrated that chimeras of DNA Polθ and family X polymerases could be engineered to gain robust terminal transferase activity and become competent for methods of nucleic acid synthesis. Therefore, all motifs, regions, and mutations demonstrated in this patent can be trivially extended to modified A family polymerases, especially DNA Polθ, to enable modified A family polymerases to incorporate 3′-modified nucleotides, reversibly terminated nucleotides, and modified nucleotides in general to effect methods of nucleic acid synthesis.

### DETAILED DESCRIPTION OF THE INVENTION

Described herein are modified terminal deoxynucleotidyl transferase (TdT) enzymes. Terminal transferase enzymes are ubiquitous in nature and are present in many species. Many known TdT sequences have been reported in the NCBI database. The sequences described herein are modified from the sequence of the Spotted Gar, but the corresponding changes can be introduced into the homologous sequences from other species. Homologous amino acid sequences of Polµ, Polβ, Polλ, and Polθ or the homologous amino acid sequence of X family polymerases also possess terminal transferase activity. References to terminal transferase also include homologous amino acid sequences of Polµ, Polβ, Polλ, and Polθ or the homologous amino acid sequence of X family polymerases where such sequences possess terminal transferase activity.

Disclosed herein is a modified terminal deoxynucleotidyl transferase (TdT) enzyme comprising at least one amino acid modification when compared to a wild type sequence, wherein the modification is selected from one or more of the amino acid positions T160, E174, C179, M183, A195, S245, H263, L265, L285, A293, D368, E385, M387, D388, K392, F394, K401, P422, E441, R442, K453, N458 or D488 of the sequence of SEQ ID NO 1 or the homologous regions in other species or a truncated portion thereof.

Disclosed herein is a modified terminal deoxynucleotidyl transferase (TdT) enzyme comprising at least one amino acid modification when compared to a wild type sequence, wherein the modification is selected from one or more of the amino acid positions T160, E174, C179, M183, A195, S198, D210, Q211, Q224, S245, R259, H263, L265, A273, H275, L285, A293, G303, Q304, L312, A314, C331, V335, M344, V348, R357, D368, I369, E385, M387, D388, F390, K392, F394, K401, A404, P422, V424, E441, R442, R445, K453, N458, K464, or D488 of the sequence of SEQ ID NO 1 or the homologous regions in other species or a truncated portion thereof.

Described herein is a modified terminal deoxynucleotidyl transferase (TdT) enzyme comprising at least the sequence ID:

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

or the equivalent homologous region in other species, wherein the sequence has one or more amino acid modifications in one or more of the amino acid positions T160, E174, C179, M183, A195, S245, H263, L265, L285, A293, D368, E385, M387, D388, K392, F394, K401, P422, E441, R442, K453, N458 or D488 of the full length sequence. The sequence above of 355 amino acids can be attached to other amino acids without affecting the function of the enzyme. For example there can be a further N-terminal sequence that is incorporated simply as a protease cleavage site, for example the sequence MENLYFQG.

Described herein is a modified terminal deoxynucleotidyl transferase (TdT) enzyme comprising at least the sequence ID:

TVSQYACQRRTTMENHNQIFTDAFAILAENAEFNESEGPCLAFMRAASLL
KSLPHAISSSKDLEGLPCLGDQTKAVIEDILEYGQCSKVQDVLCDDRYQT
IKLFTSVFGVGLKTAEKWYRKGFHSLEEVQADNAIHFTKMQKAGFLYYDD
ISAAVCKAEAQAIGQIVEETVRLIAPDAIVTLTGGFRRGKECGHDVDFLI
TTPEMGKEVWLLNRLINRLQNQGILLYYDIVESTFDKTRLPCRKFEAMDH
FQKCFAIIKLKKELAAGRVQKDWKAIRVDFVAPPVDNFAFALLGWTGSRQ
FERDLRRFARHERKMLLDNHALYDKTKKIFLPAKTEEDIFAHLGLDYIDP
WQRNA

or the equivalent homologous region in other species, wherein the sequence has one or more amino acid modifications in one or more of the amino acid positions T160, E174, C179, M183, A195, S198, D210, Q211, Q224, S245, R259, H263, L265, A273, H275, L285, A293, G303, Q304, L312, A314, C331, V335, M344, V348, R357, D368, I369, E385, M387, D388, F390, K392, F394, K401, A404, P422, V424, E441, R442, R445, K453, N458, K464, or D488 of the full length sequence. The sequence aboveof 355 amino acids can be attached to other amino acids without affecting the function of the enzyme. For example there can be a further N-terminal sequence that is incorporated simply as a protease cleavage site, for example the sequence MENLYFQG.

Disclosed is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid positions T160, E174, C179, M183, A195, S245, H263, L265, L285, A293, D368, E385, M387, D388, K392, F394, K401, P422, E441, R442, K453, N458 or D488 of the sequence of SEQ ID NO 1 or the homologous regions in other species.

Disclosed is a modified terminal deoxynucleotidyl transferase (TdT) enzyme comprising at least one amino acid modification when compared to a wild type sequence SEQ ID NO 1 or the homologous amino acid sequence of a terminal deoxynucleotidyl transferase (TdT) enzyme in other species, wherein the modification is selected from one or more of the amino acid positions T160, E174, C179, M183, A195, S198, D210, Q211, Q224, S245, R259, H263, L265, A273, H275, L285, A293, G303, Q304, L312, A314, C331, V335, M344, V348, R357, D368, I369, E385, M387, D388, F390, K392, F394, K401, A404, P422, V424, E441, R442, R445, K453, N458, K464, or D488 of the sequence of SEQ ID NO 1 or the homologous regions in other species.

Further disclosed is a modified terminal deoxynucleotidyl transferase (TdT) enzyme comprising at least two amino acid modifications when compared to a wild type sequence SEQ ID NO 1 or the homologous amino acid sequence of a terminal deoxynucleotidyl transferase (TdT) enzyme in other species, wherein the modifications are selected from modifications at the amino acid positions T160, E174, C179, M183, A195, S245, H263, L265, L285, A293, D368, E385, M387, D388, K392, F394, K401, P422, E441, R442, K453, N458 or D488 of the sequence of SEQ ID NO 1 or the homologous region in other species.

Further disclosed is a modified terminal deoxynucleotidyl transferase (TdT) enzyme comprising at least two amino acid modifications when compared to a wild type sequence SEQ ID NO 1 or the homologous amino acid sequence of a terminal deoxynucleotidyl transferase (TdT) enzyme in other species, wherein the modifications are selected from modifications at the amino acid positions T160, E174, C179, M183, A195, S198, D210, Q211, Q224, S245, R259, H263, L265, A273, H275, L285, A293, G303, Q304, L312, A314, C331, V335, M344, V348, R357, D368, I369, E385, M387, D388, F390, K392, F394, K401, A404, P422, V424, E441, R442, R445, K453, N458, K464, or D488 of the sequence of SEQ ID NO 1 or the homologous region in other species.

The modifications can be chosen from any amino acid that differs from the wild type sequence. The amino acid can be a naturally occurring amino acid. The modified amino acid can be selected from ala, arg, asn, asp, cys, gln, glu, gly, his, ile, leu, lys, met, phe, pro, ser, thr, trp, val, and sec.

For the purposes of brevity, the modifications are further described in relation to SEQ ID NO 1, but the modifications are applicable to the sequences from other species, for example those sequences listed above having sequences in the NCBI database. The sequence modifications also apply to truncated versions of SEQ ID NO 1.

The sequences can be modified at positions in addition to those regions described. Embodiments on the invention may include for example sequences having modifications to amino acids outside the defined positions, providing those sequences retain terminal transferase activity. Embodiments of the invention may include for example sequences having truncations of amino acids outside the defined positions, providing those sequences retain terminal transferase activity. For example the sequences may be BRCT truncated as described in application WO2018215803 where amino acids are removed from the N-terminus whilst retaining or improving activity. Alterations, additions, insertions or deletions or truncations to amino acid positions outside the claimed regions are therefore within the scope of the invention, providing that the claimed regions as defined are modified as claimed. The sequences described herein refer to TdT enzymes, which are typically at least 300 amino acids in length. All sequences described herein can be seen as having at least 300 amino acids. The claims do not cover peptide fragments or sequences which do not function as terminal transferase enzymes.

Modifications disclosed herein contain at least one modification at the defined positions. In certain locations, mutations can be preferentially combined.

Specific amino acid changes can include any one of C179D, C179E, C179F, C179G, C179H, C179I, C179K, C179L, C179M, C179N, C179P, C179Q, C179R, C179T, C179V, C179W, C179Y.

Specific amino acid changes can include any one of M183A, M183C, M183E, M183F, M183G, M183H, M183I, M183K, M183L, M183M, M183N, M183P, M183Q, M183S, M183T, M183V, M183W, M183Y.

Specific amino acid changes can include any one of E441A, E441C, E441D, E441F, E441G, E441H, E441I, E441K, E441L, E441M, E441N, E441P, E441Q, E441R, E441S, E441T, E441V, E441W, E441Y.

Specific amino acid changes can include any one of N458A, N458C, N458D, N458E, N458F, N458G, N458H, N458I, N458K, N458L, N458M, N458N, N458P, N458Q, N458S, N458T, N458V, N458W and/or N458Y.

Specific amino acid changes can include any one of D488A, D488C, D488E, D488F, D488G, D488H, D488K, D488I, D488L, D488M, D488N, D488Q, D488R, D488S, D488T, D488V, D488W, D488Y.

Further specific amino acid changes include P422S, P422V, P422C, P422A, P422T, P422I.

Further specific amino acid changes include R442Q, R442H.

Further specific changes include T160R, C179T,C179A, A195S, A195T, A293V

Combinations of changes may include
P422S & R442Q
P422V & R442Q
P422C & R442Q
P422V & R442H
P422A & R442H
P422T & R442H
P422T & R442Q
P422C & P442H
P422S & R442H
P422I & R442H

Specific changes may include positions L265 (P, F, V), K392M, H263 (R, Q, K), and S245(G, P).

Specific changes may include
L265P
K392M
L265P & K392M
S245G
K401T
E385D
E385N
E174S
H263R
E174S & H263R
L285M
K453N
C179E
C179S
C179G
M183L
M183Q
M183E
M183C
M183N
D349A
D349V
E441C
N458E
D488Q
F394W
D368K
D368H
D368R

Specific changes may include
M152T
T160R
E174S
C179A
C179E
C179S
C179G
C179T
M183L
M183Q
M183E
M183C
M183N
A195S
A195T
S198N
D210V
Q211R
Q224L
S245G
H263R
H263L
L265P
A273G
R259H
H275Q
L285M
G303S
Q304L
L312Q
A314S
I318L
G328A
C331R
C331Y
V335C
V335A
M344V
V348H
D349A
D349V
R357M
D368K
D368H
D368R
C381S
E385D
E385N
F390Y
K392M
F394W
K401T
A404V
P422G
P422S
V424F
V424I
E441C
R442Q
R445H
K453N
N458E
Y462F
K464T
D488Q
L265P & K392M
E174S & H263R

Specific amino acid changes include one or more of a modification selected from E174S, C179E, C179G, M183L, M183Q, M183E, M183C, M183N, S245G, S245P, H263R, H263Q, H263K, L265P, L265V, L285M, D368K, D368R, E385D,K392M, K401T, P422S, P422V, P422T, P422I, E441C, R442Q, R442H, K453N, N458E, D488Q, D488V or D488A.

Specific amino acid changes include one or more of a modification selected from S198N, D210V, Q211R, Q224L, R259H, H263L, A273G, G303S, Q304L, L312Q, A314S, C331Y, C331R, V335A, V335C, M344V, V348H, R357M, F390Y, A404V, P422G, V424F, R445H or K464T.

Specific amino acid changes include one or more of a modification selected from E385N, P422S or R442Q. Specific amino acid changes can include each of a modification E385N, P422S and R442Q. The TdT can include further additional changes.

Specific amino acid changes include one or more of a modification selected from M152T, T160R, E174S, C179A, C179T, C179E, C179G, M183L, M183Q, M183E, M183C, M183N, A195S, A195T, S198N, D210V, Q211R, Q224L, S245G, S245P, R259H, H263L, H263R, H263Q, H263K, L265P, L265V, A273G, H275Q, L285M, A293V, G303S, Q304L, L312Q, A314S, I318L, G328A, C331Y, C331R, V335A, V335C, M344V, V348H, R357M, D368K, D368R, D368H, C381S, F390Y, K392M, K401T, A404V, V424F, V424I, E441C, R445H, K453N, N458E, Y462F, K464T, D488Q, D488V or D488A.

Amino acid changes include any two or more of those listed herein in any combination.

Amino acid changes include any two or more of C179D, C179E, C179F, C179G, C179H, C179I, C179K, C179L, C179M, C179N, C179P, C179Q, C179R, C179T, C179V, C179W, C179Y, D488A, D488C, D488E, D488F, D488G, D488H, D488K, D488I, D488L, D488M, D488N, D488Q, D488R, D488S, D488T, D488V, D488W, D488Y, E441A, E441C, E441D, E441F, E441G, E441H, E441I, E441K, E441L, E441M, E441N, E441P, E441Q, E441R, E441S, E441T, E441V, E441W, E441Y, M183A, M183C, M183E, M183F, M183G, M183H, M183I, M183K, M183L, M183M, M183N, M183P, M183Q, M183S, M183T, M183V, M183W, M183Y, N458A, N458C, N458D, N458E, N458F, N458G, N458H, N458I, N458K, N458L, N458M, N458N, N458P, N458Q, N458S, N458T, N458V, N458W and/or N458Y.

Also disclosed is a method of nucleic acid synthesis, which comprises the steps of:
(a) providing an initiator oligonucleotide;
(b) adding a 3′-blocked nucleotide to said initiator oligonucleotide in the presence of a terminal deoxynucleotidyl transferase (TdT) as defined herein;
(c) removal of all reagents from the initiator oligonucleotide;
(d) cleaving the blocking group in the presence of a cleaving agent; and
(e) removal of the cleaving agent.

The method can add greater than 1 nucleotide by repeating steps (b) to (e).

References herein to ‘nucleoside triphosphates’ refer to a molecule containing a nucleoside (i.e. a base attached to a deoxyribose or ribose sugar molecule) bound to three phosphate groups. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates, such as naturally occurring modified nucleosides and artificial nucleosides.

Therefore, references herein to ‘3′-blocked nucleotide’ include nucleoside 5′-triphosphates (e.g., dATP, dGTP, dCTP or dTTP) which have an additional group on the 3′ end which prevents further addition of nucleotides, i.e., by replacing the 3′-OH group with a protecting group.

It will be understood that references herein to ‘3′-block’, ‘3′-blocking group’ or ‘3′-protecting group’ refer to the group attached to the 3′ end of the nucleotide or nucleoside triphosphate which prevents further nucleotide addition. The present method uses reversible 3′-blocking groups which can be removed by cleavage to allow the addition of further nucleotides. By contrast, irreversible 3′-blocking groups refer to dNTPs where the 3′-OH group can neither be exposed nor uncovered by cleavage.

The 3′-blocked nucleoside can be blocked by any chemical group that can be unmasked to reveal a 3′-OH. The 3′-blocked nucleoside can be blocked by a 3′-O-azidomethyl, 3′-aminooxy, 3′-O-(N-oxime) (3′—O—N═CR₁R₂, where R₁ and R₂ are each a C1-C3 alkyl group, for example CH₃, such that the oxime can be O—N═C(CH₃)₂ (N-acetoneoxime)), 3′-O-allyl group, 3′-O-cyanoethyl, 3′-O-acetyl, 3′-O-nitrate, 3′-phosphate, 3′-O-acetyl levulinic ester, 3′-O-tert butyl dimethyl silane, 3′-O-trimethyl(silyl)ethoxymethyl, 3′-O-ortho-nitrobenzyl, and 3′-O-para-nitrobenzyl.

The 3′-blocked nucleoside can also be blocked by any chemical group that can be directly utilized in chemical ligations, such as copper-catalyzed or copper-free azide-alkyne click reactions and tetrazine-alkene click reactions. The 3′-blocked nucleotide or nucleoside triphosphate can include chemical moieties containing an azide, alkyne, alkene, and tetrazine.

References herein to ‘cleaving agent’ refer to a substance which is able to cleave the 3′-blocking group from the 3′-blocked nucleotide. In one embodiment, the cleaving agent is a chemical cleaving agent. In an alternative embodiment, the cleaving agent is an enzymatic cleaving agent. The cleaving can be done in a single step, or can be a multi-step process, for example to transform an oxime (such as for example 3′-O-(N-oxime), 3′—O—N═C(CH₃)₂, into aminooxy (O—NH₂), followed by cleaving the aminooxy to OH.

It will be understood by the person skilled in the art that the selection of cleaving agent is dependent on the type of 3′-nucleotide blocking group used. For instance, tris(2-carboxyethyl)phosphine (TCEP) or tris(hydroxypropyl)phosphine (THPP) can be used to cleave a 3′-O-azidomethyl group, palladium complexes can be used to cleave a 3′-O-allyl group, or sodium nitrite can be used to cleave a 3′-aminooxy group. Therefore, in one embodiment, the cleaving agent is selected from: tris(2-carboxyethyl)phosphine (TCEP), a palladium complex or sodium nitrite.

In one embodiment, the cleaving agent is added in the presence of a cleavage solution comprising a denaturant, such as urea, guanidinium chloride, formamide or betaine. The addition of a denaturant has the advantage of being able to disrupt any undesirable secondary structures in the DNA. In a further embodiment, the cleavage solution comprises one or more buffers. It will be understood by the person skilled in the art that the choice of buffer is dependent on the exact cleavage chemistry and cleaving agent required.

References herein to an ‘initiator oligonucleotide’ or ‘initiator sequence’ refer to a short oligonucleotide with a free 3′-end which the 3′-blocked nucleotide can be attached to. In one embodiment, the initiator sequence is a DNA initiator sequence. In an alternative embodiment, the initiator sequence is an RNA initiator sequence.

References herein to a ‘DNA initiator sequence’ refer to a small sequence of DNA which the 3′-blocked nucleotide can be attached to, i.e., DNA will be synthesised from the end of the DNA initiator sequence.

In one embodiment, the initiator sequence is between 5 and 50 nucleotides long, such as between 5 and 30 nucleotides long (i.e. between 10 and 30), in particular between 5 and 20 nucleotides long (i.e., approximately 20 nucleotides long), more particularly 5 to 15 nucleotides long, for example 10 to 15 nucleotides long, especially 12 nucleotides long.

In one embodiment, the initiator sequence is single-stranded. In an alternative embodiment, the initiator sequence is double-stranded. It will be understood by persons skilled in the art that a 3′-overhang (i.e., a free 3′-end) allows for efficient addition.

In one embodiment, the initiator sequence is immobilised on a solid support. This allows TdT and the cleaving agent to be removed (in steps (c) and (e), respectively) without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In one embodiment, the initiator sequence contains a base or base sequence recognisable by an enzyme. A base recognised by an enzyme, such as a glycosylase, may be removed to generate an abasic site which may be cleaved by chemical or enzymatic means. A base sequence may be recognised and cleaved by a restriction enzyme.

In a further embodiment, the initiator sequence is immobilised on a solid support via a chemically-cleavable linker, such as a disulfide, allyl, or azide-masked hemiaminal ether linker. Therefore, in one embodiment, the method additionally comprises extracting the resultant nucleic acid by cleaving the chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In one embodiment, the terminal deoxynucleotidyl transferase (TdT) of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc. all with appropriate counterions, such as Cl) and inorganic pyrophosphatase (e.g., the Saccharomyces cerevisiae homolog). It will be understood that the choice of buffers and salts depends on the optimal enzyme activity and stability. The use of an inorganic pyrophosphatase helps to reduce the build-up of pyrophosphate due to nucleoside triphosphate hydrolysis by TdT. Therefore, the use of an inorganic pyrophosphatase has the advantage of reducing the rate of (1) backwards reaction and (2) TdT strand dismutation.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, step (d) is performed at a temperature less than 99° C., such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, steps (c) and (e) are performed by applying a wash solution. In one embodiment, the wash solution comprises the same buffers and salts as used in the extension solution described herein. This has the advantage of allowing the wash solution to be collected after step (c) and recycled as extension solution in step (b) when the method steps are repeated.

Also disclosed is a kit comprising a terminal deoxynucleotidyl transferase (TdT) as defined herein in combination with an initiator sequence and one or more 3′-blocked nucleoside triphosphates.

The invention includes the nucleic acid sequence used to express the modified terminal transferase. Included within the invention are the codon-optimized cDNA sequences which express the modified terminal transferase. Included are the codon-optimized cDNA sequences for each of the protein variants.

The invention includes a cell line producing the modified terminal transferase.

1. A modified terminal deoxynucleotidyl transferase (TdT) enzyme comprising amino acid modifications when compared to a wild type sequence SEQ ID NO 1 or a truncated version thereof or the homologous amino acid sequence of a terminal deoxynucleotidyl transferase (TdT) enzyme in other species or the homologous amino acid sequence of Polµ, Polβ, Polλ, and Polθ of any species or the homologous amino acid sequence of X family polymerases of any species, wherein the amino acid modifications are one or more of the amino acid changes E385N, P422S or R442Q.

2. The modified terminal deoxynucleotidyl transferase (TdT) enzyme according to claim 1 wherein the modification is E385N.

3. The modified terminal deoxynucleotidyl transferase (TdT) enzyme according to claim 1 or claim 2 wherein the modification is P422S.

4. The modified terminal deoxynucleotidyl transferase (TdT) enzyme according to any one of claims 1 to 3 wherein the modification is R442Q.

5. The modified terminal deoxynucleotidyl transferase (TdT) enzyme according to any one of claims 1 to 4 wherein having a further modification selected from M152T, T160R, E174S, C179A, C179T, C179E, C179G, M183L, M183Q, M183E, M183C, M183N, A195S, A195T, S198N, D210V, Q211R, Q224L, S245G, S245P, R259H, H263L, H263R, H263Q, H263K, L265P, L265V, A273G, H275Q, L285M, A293V, G303S, Q304L, L312Q, A314S, I318L, G328A, C331Y, C331R, V335A, V335C, M344V, V348H, R357M, D368K, D368R, D368H, C381S, F390Y, K392M, K401T, A404V, V424F, V424I, E441C, R445H, K453N, N458E, Y462F, K464T, D488Q, D488V or D488A.

6. The modified terminal deoxynucleotidyl transferase (TdT) enzyme according to claim 5 wherein the modification is D368H.

7. The modified terminal deoxynucleotidyl transferase (TdT) enzyme according to any one of claims 5 or 6 wherein the modification is G328A.

8. The modified terminal deoxynucleotidyl transferase (TdT) enzyme according to any one of claims 5 to 7 wherein the modification is M152T.

9. The modified terminal deoxynucleotidyl transferase (TdT) enzyme according to any one of claims 5 to 8 wherein the modification is Y462F.

10. The modified terminal deoxynucleotidyl transferase (TdT) enzyme according to any one of claims 5 to 9 wherein the modification is C381S.

11. The modified terminal deoxynucleotidyl transferase (TdT) enzyme according to any one of claims 5 to 10 wherein the modification is I318L.

12. The modified terminal deoxynucleotidyl transferase (TdT) enzyme according to any one of claims 5 to 11 wherein the modification is A195T.

13. The modified terminal deoxynucleotidyl transferase (TdT) enzyme according to any one of claims 5 to 12 wherein the modification is V424I.

14. The modified terminal deoxynucleotidyl transferase (TdT) enzyme according to any one of claims 5 to 13 wherein the modification is H275Q.

15. The modified terminal deoxynucleotidyl transferase (TdT) enzyme according to any one of claims 5 to 14 wherein the modification is C179A.

16. The modified terminal deoxynucleotidyl transferase (TdT) enzyme according to any one of claims 1 to 15 wherein the enzyme is truncated.

17. A modified terminal deoxynucleotidyl transferase (TdT) enzyme according to any one of claims 1 to 16 wherein the wild type sequence is selected from

| | |
|---|---|
| gi\|768 | Bos taurus |
| gi\|460163 | Gallus gallus |
| gi\|494987 | Xenopus laevis |
| gi\|1354475 | Oncorhynchus mykiss |
| gi\|2149634 | Monodelphis domestica |
| gi\|12802441 | Mus musculus |
| gi\|28852989 | Ambystoma mexicanum |
| gi\|38603668 | Takifugu rubripes |
| gi\|40037389 | Raja eglanteria |
| gi\|40218593 | Ginglymostoma cirratum |
| gi\|46369889 | Danio rerio |
| gi\|73998101 | Canis lupus familiaris |
| gi\|139001476 | Lemur catta |
| gi\|139001490 | Microcebus murinus |
| gi\|139001511 | Otolemur garnettii |
| gi\|148708614 | Mus musculus |
| gi\|149040157 | Rattus norvegicus |
| gi\|149704611 | Equus caballus |
| gi\|164451472 | Bos taurus |
| gi\|169642654 | Xenopus (Silurana) tropicalis |
| gi\|291394899 | Oryctolagus cuniculus |
| gi\|291404551 | Oryctolagus cuniculus |
| gi\|301763246 | Ailuropoda melanoleuca |
| gi\|311271684 | Sus scrofa |
| gi\|327280070 | Anolis carolinensis |
| gi\|334313404 | Monodelphis domestica |
| gi\|344274915 | Loxodonta africana |
| gi\|345330196 | Ornithorhynchus anatinus |
| gi\|348588114 | Cavia porcellus |
| gi\|351697151 | Heterocephalus glaber |
| gi\|355562663 | Macaca mulatta |
| gi\|395501816 | Sarcophilus harrisii |
| gi\|395508711 | Sarcophilus harrisii |
| gi\|395850042 | Otolemur garnettii |
| gi\|397467153 | Pan paniscus |
| gi\|403278452 | Saimiri boliviensis boliviensis |
| gi\|410903980 | Takifugu rubripes |
| gi\|410975770 | Felis catus |
| gi\|432092624 | Myotis davidii |
| gi\|432113117 | Myotis davidii |
| gi\|444708211 | Tupaia chinensis |
| gi\|460417122 | Pleurodeles waltl |
| gi\|466001476 | Orcinus orca |
| gi\|471358897 | Trichechus manatus latirostris |
| gi\|478507321 | Ceratotherium simum simum |
| gi\|478528402 | Ceratotherium simum simum |
| gi\|488530524 | Dasypus novemcinctus |
| gi\|499037612 | Maylandia zebra |
| gi\|504135178 | Ochotona princeps |
| gi\|505844004 | Sorex araneus |
| gi\|505845913 | Sorex araneus |
| gi\|507537868 | Jaculus jaculus |
| gi\|507572662 | Jaculus jaculus |
| gi\|507622751 | Octodon degus |
| gi\|507640406 | Echinops telfairi |
| gi\|507669049 | Echinops telfairi |
| gi\|507930719 | Condylura cristata |
| gi\|507940587 | Condylura cristata |
| gi\|511850623 | Mustela putorius furo |
| gi\|512856623 | Xenopus (Silurana) tropicalis |
| gi\|512952456 | Heterocephalus glaber |
| gi\|524918754 | Mesocricetus auratus |
| gi\|527251632 | Melopsittacus undulatus |
| gi\|528493137 | Danio rerio |
| gi\|528493139 | Danio rerio |
| gi\|529438486 | Falco peregrinus |
| gi\|530565557 | Chrysemys picta bellii |
| gi\|532017142 | Microtus ochrogaster |
| gi\|532099471 | Ictidomys tridecemlineatus |
| gi\|533166077 | Chinchilla lanigera |
| gi\|533189443 | Chinchilla lanigera |
| gi\|537205041 | Cricetulus griseus |
| gi\|537263119 | Cricetulus griseus |
| gi\|543247043 | Geospiza fortis |
| gi\|543351492 | Pseudopodoces humilis |
| gi\|543731985 | Columba livia |
| gi\|544420267 | Macaca fascicularis |
| gi\|545193630 | Equus caballus |
| gi\|548384565 | Pundamilia nyererei |
| gi\|551487466 | Xiphophorus maculatus |
| gi\|551523268 | Xiphophorus maculatus |
| gi\|554582962 | Myotis brandtii |
| gi\|554588252 | Myotis brandtii |
| gi\|556778822 | Pantholops hodgsonii |
| gi\|556990133 | Latimeria chalumnae |
| gi\|557297894 | Alligator sinensis |
| gi\|558116760 | Pelodiscus sinensis |
| gi\|558207237 | Myotis lucifugus |
| gi\|560895997 | Camelus ferus |
| gi\|560897502 | Camelus ferus |
| gi\|562857949 | Tupaia chinensis |
| gi\|562876575 | Tupaia chinensis |
| gi\|564229057 | Alligator mississippiensis |
| gi\|564236372 | Alligator mississippiensis |
| gi\|564384286 | Rattus norvegicus |

.

18. A method of nucleic acid synthesis, which comprises the steps of:
(a) providing an initiator oligonucleotide;
(b) adding a 3′-blocked nucleotide to said initiator oligonucleotide in the presence of a terminal deoxynucleotidyl transferase (TdT) as defined in any one of claims 1 to 17;
(c) removal of all reagents from the initiator oligonucleotide;
(d) cleaving the blocking group in the presence of a cleaving agent; and
(e) removal of the cleaving agent.

19. The method as defined in claim 18, wherein greater than 1 nucleotide is added by repeating steps (b) to (e).

20. The method as defined in claim 18 or claim 19 wherein the 3′-blocked nucleotide is blocked with a group selected from 3′-O-azidomethyl, 3′-aminooxy, 3′-O-(N-oxime), 3′-O-allyl 3′-O-cyanoethyl, 3′-O-acetyl, 3′-O-nitrate, 3′-phosphate, 3′-O-acetyl levulinic ester, 3′-O-tert butyl dimethyl silane, 3′-O-trimethyl(silyl)ethoxymethyl, 3′-O-ortho-nitrobenzyl, or 3′-O-para-nitrobenzyl.

21. The method as defined in claim 20 wherein the 3′-blocked nucleoside is blocked by either a 3′-O-azidomethyl, 3′-aminooxy or 3′-O-allyl group.

22. A kit comprising a terminal deoxynucleotidyl transferase (TdT) as defined in any one of claims 1 to 17 in combination with an initiator oligonucleotide and one or more 3′-blocked nucleoside triphosphates.
